(19) United States
(12) Patent Application Publication
(10) Pub. No.: US 2015/0223681 A1
(43) Pub. Date: Aug. 13, 2015

(54) Method and Apparatus for Ultrafast Multi-Wavelength Photothermal Optical Coherence Tomography (OCT)

(71) Applicant: The Board of Regents of the University of Texas System, Austin, TX (US)

(72) Inventors: Roman V. Kuranov, San Antonio, TX (US); Austin B. McElroy, Austin, TX (US); Jordon Dwelle, Austin, TX (US); Thomas E. Milner, Austin, TX (US)

(73) Assignee: The Board of Regents of the University of Texas Systems, Austin, TX (US)

(21) Appl. No.: 14/425,038

(22) PCT Filed: Aug. 30, 2013
(86) PCT No.: PCT/US13/57534

§ 371 Date: Feb. 28, 2015

Related U.S. Application Data
(60) Provisional application No. 61/695,072, filed on Aug. 30, 2012.

(51) Int. Cl. A61B 3/10; G01B 9/02; A61B 5/145; A61B 5/1455; A61B 5/00; A61B 5/02

(52) U.S. Cl. CPC A61B 3/102; A61B 5/7257; A61B 5/444; A61B 5/02007; A61B 5/4331; A61B 5/4381; A61B 5/4238; A61B 5/4255; A61B 5/4233; A61B 5/4552; A61B 5/4064; A61B 5/14546; A61B 5/14552; G01B 9/02091; G01B 9/02084

(57) ABSTRACT

Certain embodiments are directed to methods and apparatus for ultrafast MWP-OCT that is based on precise control of the OCT tunable-laser source combined with synchronization of the photothermal excitation laser emission. The invention is generally related to physics, biology, medicine, and imaging. In certain embodiments the field of the invention is directed to multiple wavelength photothermal optical coherence tomography.

The invention is generally related to physics, biology, medicine, and imaging. In certain embodiments the field of the invention is directed to multiple wavelength photothermal optical coherence tomography.

Optical Coherence Tomography (OCT) is a high-resolution optical tomography technique. This technique is capable of realizing a high resolution (approximately 1 to 10 μm), close to the optical wavelength, by employing optical interference phenomenon. Furthermore, a probe used to capture a tomographic image is an optical probe, and therefore X-ray exposure does not pose a problem, in contrast to X-ray Computed Tomography (CT). Using these high resolution and non-invasive qualities, diagnostic apparatus for observing the posterior of the eye and the anterior eye portion at a high resolution on a par with a microscope can be constructed.

Non-invasive analysis is a valuable technique for acquiring information about systems or targets without undesirable side effects, such as damaging or contaminating the system being analyzed. In the case of analyzing living entities, such as human tissue, undesirable side effects of invasive analysis include the risk of infection along with pain and discomfort associated with the invasive process. In measuring components in the blood, it is highly desirable for example to measure the oxygenation level in blood frequently and accurately to provide appropriate treatment. A non-invasive method would avoid the pain and risk of infection and provide an opportunity for frequent or continuous measurement.

Non-invasive analysis techniques have been proposed. These techniques include: near infrared spectroscopy using both transmission and reflectance; spatially resolved diffuse reflectance; frequency domain reflectance; fluorescence spectroscopy; polarimetry; and Raman spectroscopy. These techniques are vulnerable to inaccuracies due to issues such as, environmental changes, presence of varying amounts of interfering contamination, skin heterogeneity, and variation of location of analysis. These techniques also require considerable processing to de-convolve the required measurement, typically using multi-variate analysis and have typically produced insufficient accuracy and reliability for an intended application.

There is a need for additional methods and apparatus for commercially viable, robust, non-invasive devices with ability to measure and detect components in a target, such as, but not limited to human tissue.

### SUMMARY

Optical Coherence Tomography (OCT) is an imaging modality that provides a high-resolution image in scattering media, such as tissue. OCT becomes an imaging modality of choice for a wide range of medical and other applications where detailed microstructural information is of interest. OCT provides unprecedented high-resolution structural information that can benefit from ultrafast acquisition of the signals required for OCT imaging.

Ultrafast optical coherence tomography irradiates a target with an OCT probe beam having multiple optical frequencies (νᵢ). Each optical frequency (νᵢ) can be substantially fixed or has a narrow bandwidth with a fixed central frequency. A substantially fixed optical frequency corresponds to an OCT probe beam in which variation in optical frequency (while the target is irradiated by each optical frequency (νᵢ) in the OCT probe beam) results in a signal phase variation less than a target value (see Eq. 5 below). For example, when using an akinetic tunable laser source (e.g., Insight Photonics) variation in the optical frequency can be less than 30 MHz during the time (2.5 ns) while the target is irradiated by each optical frequency (νᵢ) in the OCT probe beam so that signal phase variation is less than 0.1 radians at a 4 mm depth.

A sample can be irradiated with an OCT probe beam at a particular frequency or frequencies for up to 5 microseconds. In certain aspects the target is irradiated by an OCT probe beam having an optical frequency νᵢ for at least, at most, or about 1, 10, 20, 30, 40, 50, 60, 70, 80, 90, 100, 200, 300, 400, 500, 600, 700, 800, or 900 picoseconds (ps) or nanoseconds (ns) including all values and ranges there between. In a further aspect, the target is irradiated at an optical frequency (νᵢ) in the OCT probe beam for at least, at most, or about 0.001, 0.01, 0.1, 1, 2, 3, 4, up to 5 microseconds, including any value or range there between. In certain aspects, the OCT probe beam is a narrow bandwidth light beam with a fixed central frequency.

In certain aspects, a plurality of time resolved narrow bandwidth OCT probe beams are used. In certain aspects, 100, 500, 600, 700, 800, 900, 1000, 2000, 3000, 4000, 5000, 10,000, or more distinct OCT probe beam with substantially fixed frequencies (νᵢ) can be used. In a further aspect, the narrow bandwidth probe beams central frequencies group around central mass optical frequency (OCT working frequency, ν̅=c/ λ̅ where c is speed of light in a vacuum and λ is wavelength) so the Fourier or other transformation for conjugating frequency with time domain-space (i.e., wavenumber with in depth distance space, z) gives an OCT depth profile (A-scan). The term “A-line” or “A-scan” refers to an axial scan relative to OCT probe beam propagation (a line along the depth or z axis direction). The term “B-line” or “B-scan” refers to a collection of a number of A-scans captured when a probe beam scans over a target in the lateral or x-direction. “C-scan” refers to a collection of a number of B-scans captured when the probe beam scans over a sample in an elevational or y-direction. In certain aspects 500, 1000, 5000, 10,000, 50,000, 100,000, 500,000, 1,000,000 or more A-scans are acquired in less than 0.001, 0.01, 0.1, 1, 2, 3, 4, 5, 6, 7, 8, 9, or 10 milliseconds or seconds.

Certain embodiments are directed to methods for Fourier-Domain optical coherence tomography. Slower and lower signal-to-noise-ratio (SNR) time-domain OCT axial scans (A-scan) achieved by effective modulation of optical pathlength between reference and sample OCT interferometer arms. A simple way of optical pathlength modulation is linear translation of a mirror in the reference arm of the interferometer. No Fourier transformation of the data is needed in time-domain OCT. In the Fourier-Domain OCT, pathlength in the OCT interferometer arms is normally fixed during A-scan acquisition. In Fourier domain OCT, depth information in an A-scan is achieved simultaneously from all imaging depths by acquiring the interference fringe intensity at a variety of optical frequencies (νᵢ, spectral information) and subsequent Fourier transformation of the spectrum of interference fringe intensities. There are two different approaches to acquire spectrally resolved interference fringe intensities: (a) OCT light source optical frequency is tuned over a desired spectral range and a single photodetector records spectrum of the interference fringe intensity in time—Swept-Source OCT; and (b) irradiation with broadband light and using a diffraction grating in the detection arm of the OCT interferometer to spatially/angularly separate different optical frequencies. In this case, the interference fringe intensity spectrum is detected by a linear array of detectors before Fourier Transformation, each detector recording the interference fringe intensity for a specific optical frequency—Spectral-Domain OCT (SD-OCT). In certain aspects the methods comprise (a) exposing a first location of a target to a plurality of time resolved, narrow bandwidth optical coherence tomography (OCT) probe beams having substantially fixed optical frequencies and an exposure time interval of at most 5 microseconds; (b) acquiring an interferometric signal related to each substantially fixed optical frequency OCT probe beam (νᵢ); (c) processing the acquired signals to produce an A-scan; and (d) repeating steps a-c to produce a plurality A-scans representative of the target.

In certain aspects, acquisition of signal can be performed by a single detector at different times (e.g., swept-source based OCT) or by two or more detectors at the same or different times from a spatially distributed one-dimensional or two-dimensional detector array (spectrometer based OCT or SD-OCT). In SD-OCT the light at different frequencies can be spatially distributed by a dispersive element, such as a grating.

Certain embodiments are directed to methods for constructing an optical coherence tomography probe beam. In certain aspects the methods comprise (a) directing a light beam from a broadband pulsed optical source to a system of spatially, temporally, or dynamically changing narrow bandwidth reflectors to provide a plurality of spatially resolved beams having a distinct fixed central optical frequency (spatially changing narrow bandwidth reflectors implementation is shown in FIG. 1); (b) directing the broadband beam through a dispersive optical system so the different optical frequencies travel with different speed for a fixed distance and acquire different time delays and next directing the time-broadened beam through a comb-like optical filter (high-absorption frequency regions interlays with high-transmission frequency ranges). In both cases the initial broadband short pulse is converted to a narrow bandwidth pulse train. That pulse train can be used as an optical source for ultra-fast Photothermal OCT in analogy with the existing akinetic swept source (Insight Photonic Solutions Inc.) as described below.

The OCT probe beams described herein can be used in combination with excitation beams to interrogate various components in a target region. In certain aspects, components of a target will interact with various frequencies of probe beam, which can be detected and used for a variety of purposes, e.g., measuring the levels of one or more component, or measuring the relative concentrations of two or more components.

As used herein the term “excitation,” unless otherwise indicated, refers to the photothermal excitation produced by the absorption of radiation.

The use of the word “a” or “an” when used in conjunction with the term “comprising” in the claims and/or the specification may mean “one,” but it is also consistent with the meaning of “one or more,” “at least one,” and “one or more than one.”

It is contemplated that any embodiment discussed herein can be implemented with respect to any method or composition of the invention, and vice versa. Furthermore, compositions and kits of the invention can be used to achieve methods of the invention.

Throughout this application, the term “about” is used to indicate that a value includes the standard deviation of error for the device or method being employed to determine the value.

The use of the term “or” in the claims is used to mean “and/or” unless explicitly indicated to refer to alternatives only or the alternatives are mutually exclusive, although the disclosure supports a definition that refers to only alternatives and “and/or.” It is also contemplated that anything listed using the term “or” may also be specifically excluded.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### DESCRIPTION OF THE DRAWINGS

The following drawings form part of the present specification and are included to further demonstrate certain aspects of the present invention. The invention may be better understood by reference to one or more of these drawings in combination with the detailed description of the specification embodiments presented herein.

FIG. 1. Illustration of one embodiment of an OCT light source to provide a probe beam for ultra-fast Photothermal or Fourier-Domain OCT. Initial source is a broadband (5-200 nm bandwidth) pulsed laser featuring short input pulse (femtosecond or picosecond range) that is transformed to a train of longer (microseconds to picoseconds) narrow (subpicometer to few nanometers range) line pulses. Pulses have different optical frequencies (νᵢ) and delayed between each other for a known time, e.g., 2 ns. The amplitudes of the narrow pulses can be selected as desired, for example, maximum amplitude in the center and amplitude decreases to the edges of pulse train. The narrow pulse frequency determined by narrow bandwidth mirror (WM), the amplitude by the combination of broadband source spectrum and reflectivity of the associated specific WM. Such a delay line with spectral mirrors can be a fiber with fiber bragg grating that are separated along the fiber.

FIG. 2. An illustration of a general principle of photothermal optical coherence tomography.

FIG. 3. An illustration of timing pattern of laser photothermal excitation (of tissue chromophores) and OCT emission (lower rectangle) profiles. The OCT laser emits at a substantially fixed optical frequency (νᵢ) over some period of time that is divided into pre-pulse (a) and post-pulse OCT signals (b). Excitation laser light is selectively absorbed by target chromophores. OCT light may have the same polarization state for both pre- and post-time segments (A) or polarization for post-pulsed OCT light can be orthogonal to the pre-pulsed OCT light (B). Photothermal excitation light can target one chromophore (FIGS. 3A and 3B) or two and more chromophores (FIGS. 3C and 3D). When targeting one chromophore one or more photothermal excitation light wavelengths is/are used, when targeting two or more chromophores at least two excitation wavelengths are used. A single photothermal excitation wavelength per each OCT emission frequency can be used (C) and two or more photothermal excitation wavelengths per OCT emission frequency (D) (FIG. 3A-3C). Note for case C that νᵢ₋₁=νᵢ and νᵢ₊₁=νᵢ₊₂ is possible. In the last case (FIG. 3D) region “a” is pre- and region “b” is post-pulse for first photothermal excitation wavelength and region “b” is pre- and region “c” is post-pulse for the second photothermal excitation wavelength and so on.

FIG. 4. An illustration of pre-pulse and post-pulse OCT signal fringes in two different OCT detection channels.

FIG. 5. An illustration of synchronization of photothermal excitation light source with pre- and post-pulse OCT detection channels. In this approach two photo-detectors and two ADC's (ADC1 and ADC 2) are utilized to acquire pre- and post-pulse OCT signals, respectively. This synchronization scheme may be applied for excitation patterns illustrated in FIGS. 3A, 3B and 3C. For the excitation pattern illustrated in FIG. 3D (i.e., two photothermal excitation beams for each OCT probe wavelength) the beam splitter should provide 3 or more beams and the number of ADCs (n_{ADC}) corresponds to the number of beams: n_{ADC}=nₚₑ+1 where nₚₑ is a number of photothermal excitation wavelengths in FIG. 3D. Variable delay element can utilize optical or/and electrical delaying parts.

FIG. 6. An illustration of synchronization of photothermal excitation light source with pre- and post-pulse OCT detection channels. In this approach one photo-detector and two ADCs (ADC1 and ADC2) are utilized to acquire pre- and post-pulse OCT signals. ADC1 and ADC2 are triggered to acquire the pre- and post-pulse OCT interference fringe signals. This synchronization scheme is valid for excitation patterns illustrated in FIGS. 3A, 3B and 3C. For the excitation pattern illustrated in FIG. 3D the number of ADC channels: n_{ADC}=nₚₑ+1, where nₚₑ is a number of photothermal excitation wavelengths.

FIG. 7. An illustration of synchronization of photothermal excitation light source using non-polarizing beam splitter and balanced detector. In this approach one balanced photo-detector and two ADCs (ADC1 and ADC2) are utilized to acquire difference between pre- and post-pulse OCT interference fringe signals. An optical delay line introduces a delay between pre- and post-pulse signals. This synchronization scheme is valid for excitation patterns illustrated in FIGS. 3A, 3B, and 3C. When multiple photothermal excitation pulses are utilized (FIG. 3D) the apparatus depicted can be modified to acquire the appropriate signals.

FIG. 8. An illustration of pre- and post-pulse OCT interference fringe signal subtraction using differential channel of the balanced detector in FIG. 7. Bar after balanced detection represents differential OCT interference fringe signal between pre- and post-pulse times.

FIG. 9. An illustration of synchronization of photothermal excitation light source using polarizing beam splitter and a balanced detector. This synchronization scheme is valid for excitation patterns illustrated in FIGS. 3A, 3B, and 3C.

FIG. 10. An example of producing the polarization split in FIG. 9. Light from tunable-laser goes through the polarization controller (PC) and polarizer (P) to ensure a high degree of polarized light. Light is divided into two orthogonally polarized beams using a polarization beam splitter (PBS). The orthogonally polarized beams delayed with respect to each other for the desired time period (1 ns equals 30 cm of the optical path difference). Precise control of the time delay may be achieved using an optical delay line. Polarization of one or both beams can be adjusted using a polarization controller and the light recombined in the non-polarized beam splitter (BS). As a result light that has two orthogonally polarized components with a desired time delay enter the OCT interferometer. A polarization split scheme can be implemented using polarization-maintaining bulk optic elements or combination of those optical elements.

FIGS. 11A and 11B. An example of producing the two (A) and arbitrary (B) delayed pulses at selected wavelengths using a broadband pulsed laser (for example femtosecond laser). BS—beam splitter, F_{λn}—filter to select specified spectrum around desired wavelength (λ, n), DLₙ—nth delay line.

FIG. 12. Ultra-fast SO₂-OCT (A) and wavelength separation item of the Multi-Wavelength Photothermal (MWP)-OCT (B). The femto-second laser, say with repetition rate of 75 MHz used as a source of photothermal excitation light. Wavelength separation unit provides two delayed pulses, say at 770 nm and 800 nm. The pulse duration determined by the filters (F₇₇₀ and F₈₀₀) spectra is 1 ps with 3 ns delay between them. Synchronization scheme employed can be with any other OCT probe and photothermal excitation light sources.

FIG. 13. Laser photothermal excitation timing pattern and OCT emission profiles specific for FIG. 12. OCT tunable laser tuned at the next emission frequency (νᵢ₋₁, νᵢ) triggered by photothermal excitation light source (black dashed pulse). The sample is photothermally excited at 770 nm and 800 nm.

FIG. 14. Rat retinal B-scan (A) and inter B-scan motion (B). B-scan time duration is 36 ms. Vessels are indicated by arrows. Labels here are ‘1’ is retina, ‘2’ is choroid and ‘3’ is sclera (A).

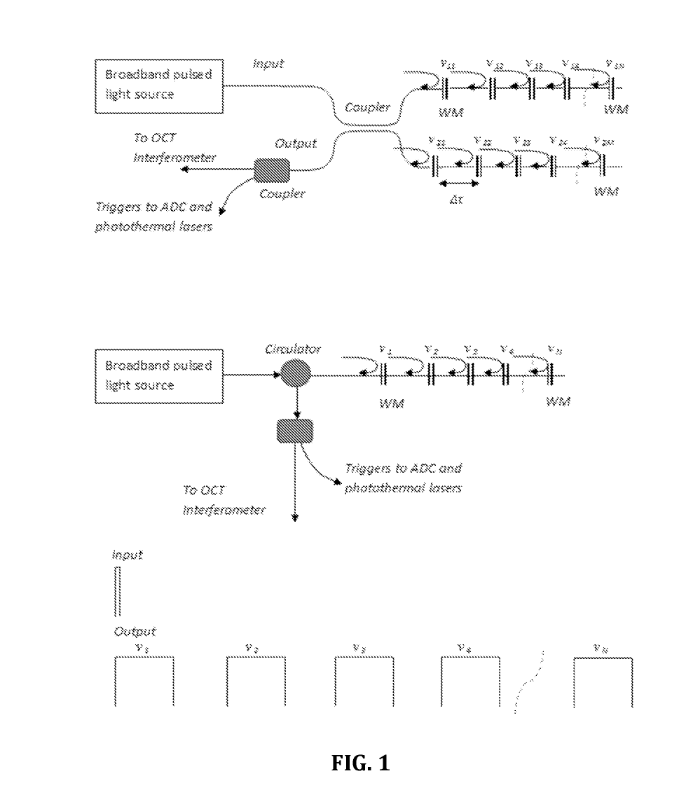

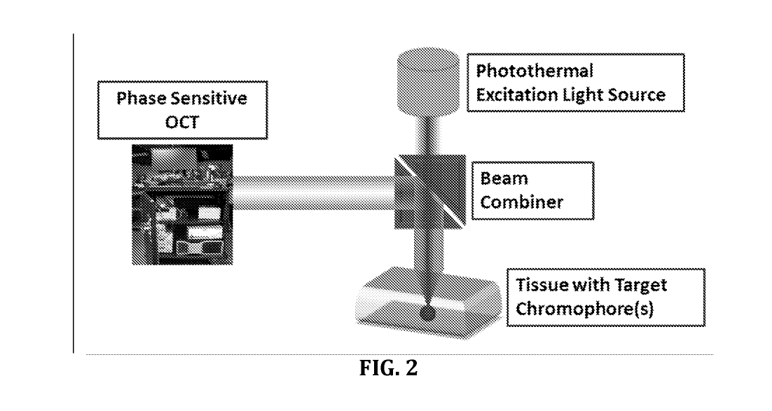

### DESCRIPTION

One challenge in translating Photothermal and Multiwavelength Photothermal (MWP)-OCT to clinical and industrial applications is the influence of physiological and environmental motion artifacts of the target sample on optical path length (op) measurements. In current practice, motion artifacts are minimized by modulating photothermal excitation light at a carrier frequency in the range from few hundred to few thousands of Hz. To provide sufficient system sensitivity, the photothermal signal is collected for a time period ranging from a few milliseconds (Zhou et al., Optics Letters, 35(5):700-02, 2010) to one hundred milliseconds (Guan et al., Journal of Biomedical Optics, 16(12):126003-09, 2011) for phantoms and ex vivo samples containing exogenous chromophores and few seconds for in vivo samples by targeting endogenous chromophores (Kuranov et al., Opt Express 19(24):23831-44, 2011). The methods described herein decrease substantially the photothermal signal collection time to determine op variation resulting from pulsed laser excitation for one A-scan to the microsecond level using available OCT tunable-laser sources. In certain aspects, sub A-scan op variation measurements can be acquired. In certain aspects, motion artifacts for OCT tunable-laser sources are negligible in the range of a few to tens of nanoseconds depending on specific signal processing architecture and procedure and number of photothermal excitation wavelengths used.

### I. Ultrafast Multi-Wavelength Photothermal OCT (UMWP-OCT)

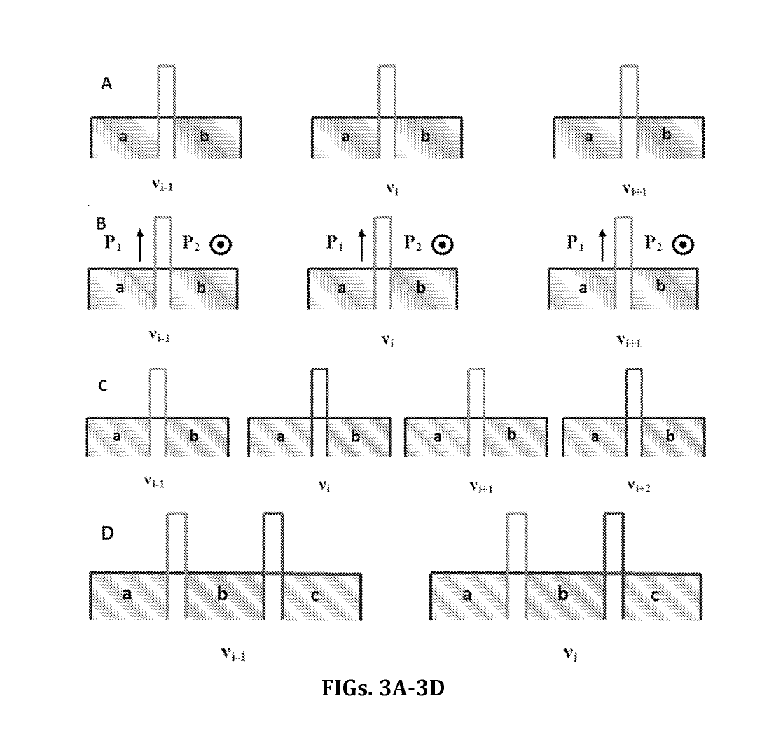

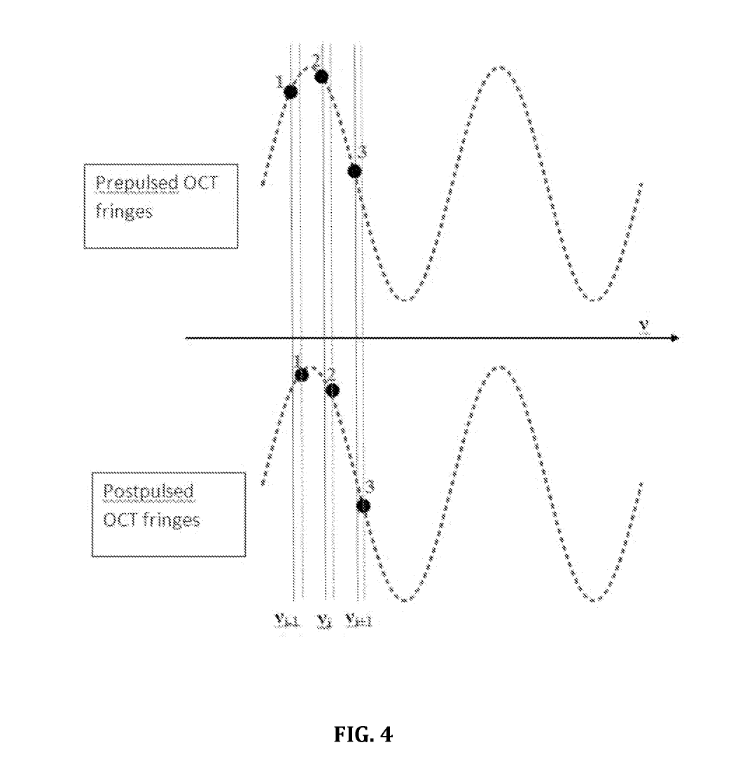

Certain embodiments are directed to methods and apparatus for ultrafast MWP-OCT that is based on precise control of the OCT laser source (OCT probe beam) combined with synchronization of the photothermal excitation light source (FIG. 3). As an example, the OCT source emits light (OCT probe beam) at a single optical frequency νᵢ₋₁ during some period of time (e.g., 2.5-5.0 ns for the Insight Photonic Solutions, Inc. source). The photothermal excitation beam is synchronized with the OCT probe beam so that the target chromophore(s) are excited when the OCT probe beam is incident on the sample at a fixed OCT optical frequency (νᵢ₋₁). The photothermal excitation beam pulse time duration is selected to be shorter than time duration of OCT probe beam emission at each optical frequency (νᵢ). The synchronization of the photothermal excitation light source and the OCT probe beam gives pre-pulse (region a in FIG. 3) and post-pulse (region b in FIG. 3) time intervals for acquisition of the OCT signal at each optical frequency (νᵢ). In certain aspects, the OCT probe beam may have the same polarization state or a different polarization state for both pre- and post-time segments (FIG. 3A) or polarization for post-pulsed OCT light can be orthogonal to the pre-pulsed OCT light (FIG. 3B). In one example, a pre-pulsed OCT probe beam can have vertical and a post-pulsed OCT probe beam can have horizontal linear polarization or the beams can have right and left circular polarizations etc.

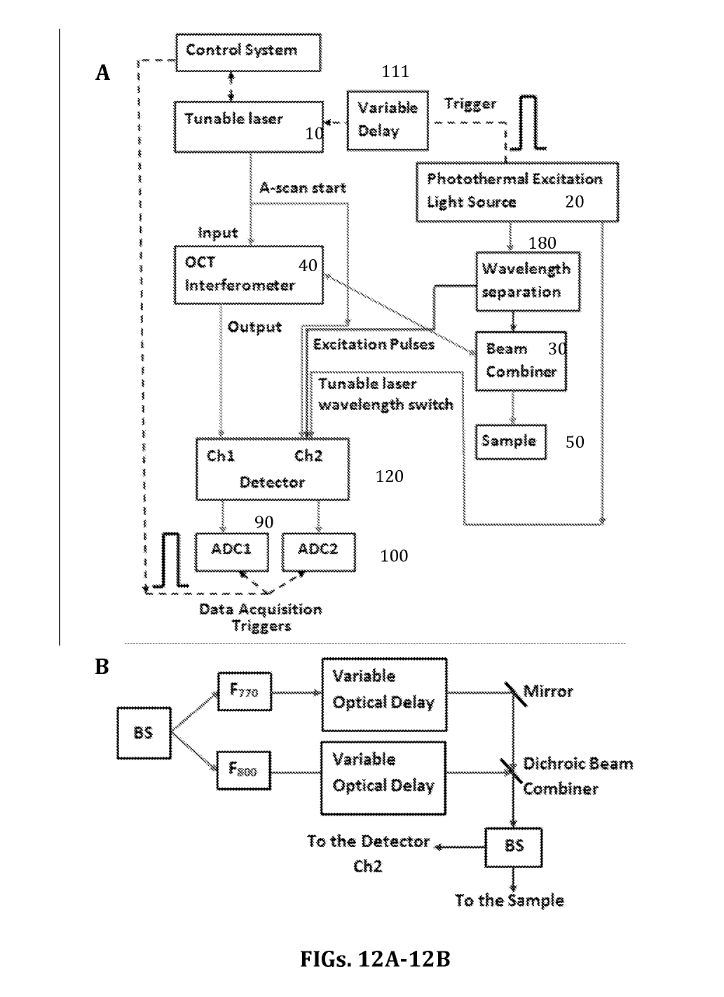

In certain embodiments, separate optical detectors record pre-pulse and post-pulse OCT interference fringe signals. Analog-to-digital converters (ADC), or a balanced detector and one ADC can be used, see below. When two ADCs are used, the pre-pulse and post-pulse OCT interference fringe signals are acquired by ADCs triggered at the same time, an optical delay can be implemented. Alternatively, an electronic trigger delay can be implemented when propagation time of the probe beam pre-pulse and post-pulse signals is different. In the case when one ADC is utilized, the arithmetic difference between pre- and post-pulse OCT interference fringe signals is detected. In certain aspects in case of one ADC with two channels (FIG. 12) one channel continuously samples the OCT interference fringe signal and the other channel continuously detects trigger events (i.e., photothermal excitation times and times of OCT probe beam such as wavelength switching and starting new A-scan). An additional control system may be implemented for triggering start and duration of data acquisition. In certain aspects and optical delay line is utilized so that pre- and post-pulsed OCT signals arrive at photodiodes within a balanced detector at the same time.

Once the pre-pulse and post-pulse OCT interference fringe signals are sampled, the OCT probe beam can be tuned to different frequency, νᵢ. The photothermal excitation source is configured to provide pre- and post-pulse OCT interference fringe signals at the frequency νᵢ.

This process of acquiring OCT interference fringe signals in pre- and post-pulse acquisition intervals continues for a number of optical frequencies (νᵢ) so that an entire optical spectrum of the OCT interference fringe signal is recorded to construct an A-scan depth profile with the desired axial resolution. Successively probed OCT A-scans and photothermal excitation beams may be moved to the next spatial location on the sample and data recorded to construct an OCT B-scan.

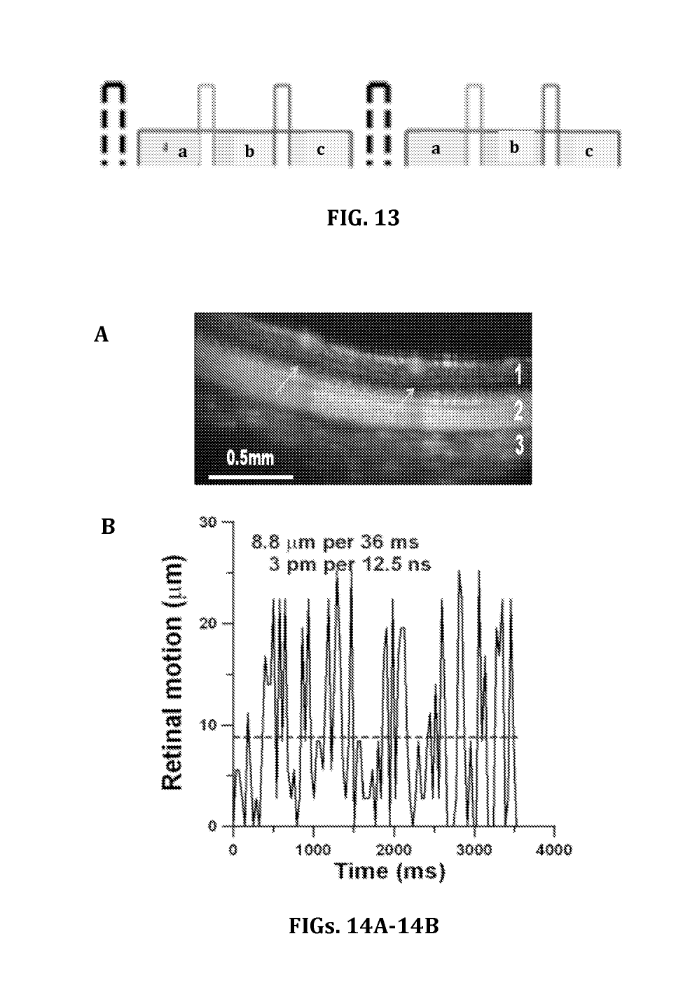

Ultra-fast SO₂-OCT fundamentals. One example of the methods and apparatus described herein includes a 75 Mhz repetition rate Ti:sapphire femtosecond laser (e.g., Mira, Coherent, Santa Clara, Calif.) providing photothermal excitation light (FIG. 12) and a tunable laser (Insight Photonic Solution, Lafayette, Colo.) capable of ultrafast (2.5 ns) optical frequency switching and emitting at the selected frequency until the next trigger event. Part of the pulse emitted by the Ti:sapphire laser is used to trigger (bold hashed lines in FIG. 13) the tunable laser frequency switch and the other part is used to create two delayed picosecond pulses at 770 nm (pulse between a and b in FIG. 13) and 800 nm (pulse between b and c in FIG. 13). For this example the broadband femtosecond pulse is divided into two beams (FIG. 12B) and narrow band (0.9 nm) 1 ps pulses at 770 nm and 800 nm are created by notch filters (F₇₇₀ and F₈₀₀). The 0.6 m optical fiber introduces a 3 ns delay of the 770 nm photothermal excitation pulse compared to the 800 nm photothermal excitation pulse (other variable optical delay lines can be used to correct timing issues). The OCT interference fringe signal is recorded by analog-to-digital-converter's (ADC, 1.6 G sample/s, NI, Austin, Tex.) channel 1 while trigger events corresponding to switching optical frequency of the tunable laser and delivery time of photothermal excitation light are recorded in channel 2. The information from channel 2 is used to sort out pre- and post-pulsed SO₂-OCT signals, which are continuously recorded in the ADC's channel 1. In FIG. 13, region “a” is pre- and region “b” is post-pulsed for 800 nm excitation, and region “b” is pre- and region “c” is post-pulsed for 770 nm excitation pulses.

Extraction of the phase difference between pre-pulse and post-pulse OCT interference fringe signals and amplitude of the OCT signal (pre- and post-pulsed amplitudes considered to be equal) can be done as described below. Extraction of the phase difference between pre- and post-pulse phase and amplitude of the OCT interference fringe signal (pre- and post-pulsed amplitudes considered to be equal) achieved by considering a retinal OCT signal. The pre- and post-pulse OCT signals may be written:

$$\begin{matrix}{S}_{pre} ( {v}_{i} ) = 2 \cdot {A}_{s} ( {v}_{i} ) \cdot cos ( \frac{2 π {v}_{i} Δ z ( {v}_{i} )}{c} + α ) , \\ {A}_{s} = {A}_{sam} \cdot \sqrt{{I}_{ref}} & ( 1 ) \\ {S}_{post} ( {v}_{i} ) = 2 \cdot {A}_{s} ( {v}_{i} ) \cdot cos ( \frac{2 π {v}_{i} Δ z ( {v}_{i} )}{c} + α + δϕ ( {v}_{i} ) ) & ( 2 )\end{matrix}$$

Here δφ(νᵢ) is an OCT signal phase change induced by chromophore absorption of the photothermal excitation, Aₛₐₘ(νᵢ) is an electrical field amplitude backscattered from the retina, Δz(νᵢ) pathlength difference between sample and reference paths. I_{ref}(νᵢ)—intensity reflected back from the reference arm in the OCT interferometer at tunable laser optical frequency νᵢ, α is a constant phase depending on absolute value of the νᵢ.

Comparing the sum and difference of the signals, the photothermal phase shift between pre- and post-pulsed OCT signals (δφ) the and OCT amplitude (Aₛ(νᵢ)) can be determined:

$$\begin{matrix}δϕ ( {v}_{i} ) = 2 \sqrt{\frac{{( {S}_{pre} ( {v}_{i} ) - {S}_{post} ( {v}_{i} ) )}^{2} - {( {S}_{pre} ( {v}_{i - 1} ) - {S}_{post} ( {v}_{i - 1} ) )}^{2}}{{( {S}_{pre} ( {v}_{i} ) + {S}_{post} ( {v}_{i} ) )}^{2} - {( {S}_{pre} ( {v}_{i - 1} ) + {S}_{post} ( {v}_{i - 1} ) )}^{2}}} & ( 3 ) \\ {A}_{s} ( {v}_{i} ) = \sqrt{4 {( {S}_{pre} ( {v}_{i - 1} ) - {S}_{post} ( {v}_{i - 1} ) )}^{2} / δϕ ( {v}_{i} ) + {( {S}_{pre} ( {v}_{i - 1} ) + {S}_{post} ( {v}_{i - 1} ) )}^{2}} / 4 & ( 4 )\end{matrix}$$

This phase shift (δφ(νᵢ)) is induced by absorption of radiant energy emitted by the photothermal excitation light source by the target chromophore during one photothermal pulse. The inventors are able, therefore, to acquire amplitudes and photothermally induced phase shifts over the entire spectrum of the tunable laser source to construct both 3-D morphological and blood oxygenation maps:

$${Δϕ}_{1 , 2} ( {z}_{j} ) = 2 \cdot Arg \{ FFT [ {A}_{s} ( {v}_{i} ) \cdot cos ( \frac{2 π {v}_{i} Δ z ( {v}_{i} )}{c} + {δϕ}_{1 , 2} ( {v}_{i} ) ) ] \} - 2 \cdot Arg \{ FFT [ {A}_{s} ( {v}_{i} ) \cdot cos ( \frac{2 π {v}_{i} Δ z ( {v}_{i} )}{c} ) ] \} \\ A ( {z}_{j} ) = 2 \cdot Abs \{ FFT [ {A}_{s} ( {v}_{i} ) \cdot cos ( \frac{2 π {v}_{i} Δ z ( {v}_{i} )}{c} ) ] \} , \\ Δ z ( {v}_{i} ) = \frac{c}{2 π ( {v}_{i} - {v}_{i - 1} )} [ Arc cos ( \frac{\overline{S} ( {v}_{i - 1} )}{2 {A}_{s} ( {v}_{i - 1} )} ) - Arc cos ( \frac{\overline{S} ( {v}_{i} )}{2 {A}_{s} ( {v}_{i} )} ) ] \qquad ( 5 )$$

Here subscript 1 denotes photothermal laser excitation at λ₁=770 nm and subscript 2 at λ₂=800 nm, S̅(νᵢ)=0.5[Sₚᵣₑ(νᵢ)+Sₚₒₛₜ(νᵢ)] and, S̅(νᵢ₋₁)=0.5[Sₚᵣₑ(νᵢ₋₁)+Sₚₒₛₜ(νᵢ₋₁)]. The oxygenation maps are constructed from a 3-D op_{1,2}(zⱼ,xₘ)=Δφ_{1,2}(zⱼ,xₘ)λ/(4π) map as described in (Kuranov et al., Opt Express 19(24):23831-44, 2011) and represented in color coded format superposed on the intensity OCT images obtained from A(zⱼ,xₘ), where xₘ is the lateral position and m is lateral position index. Lateral scanning can be achieved by a standard method using galvo-mirrors steering (6210H, Cambridge Tech., Lexington, Mass.). In one example the inventors use 1,024 optical frequencies per OCT A-scan (75 kA-scan acquisition rate) and 1,000 A-scans (5 μm lateral step) per B-scan. B-scans will have 13 μm of lateral separation taking just 5 s to image 5 mm×5 mm retinal area.

Following the procedure described above, a 3D-map of both optical reflectivity (structure) and spectral absorption signature of targeted chromophore(s) (composition) can be acquired. To acquire a more detailed representation of the spectral absorption signature of tissue chromophores, the photothermal excitation light source optical frequency may be varied either from one OCT emitting optical frequency to another or from one A-scan to another depending on the phase extraction algorithm (eq. 8 and 9 or equation 10).

Certain aspects are directed to methods for constructing an optical coherence tomography probe beam comprising (a) directing a light beam from a broadband pulsed optical source to a system of spatially, temporally, or dynamically changing narrow bandwidth reflectors to provide a plurality of spatially resolved beams having a distinct fixed central frequency; (b) directing the plurality of spatially resolved beams through a dispersive optical system so the different optical frequencies travel with different speed for a fixed distance and acquire different time delays.

In a further aspect, methods are directed to Fourier-Domain photothermal optical coherence tomography comprising (a) exposing a target to a plurality of time resolved optical coherence tomography (OCT) probe beams, each probe beam having a distinct fixed frequency, and an exposure interval of at most 5 microseconds; (b) exposing the target to an excitation pulse of less than 2 microseconds during the probe beam exposure interval; (c) acquiring an interferometric signal related to each probe beam exposure interval before the excitation pulse and after the excitation pulse; and (c) processing the acquired signals. In certain aspects, the OCT interference fringe signal is acquired continuously. The method can further comprise a plurality of photothermal excitation pulses during the probe beam exposure interval and acquiring a signal before and after each excitation pulse.

### II. UMW-OCT System and Apparatus

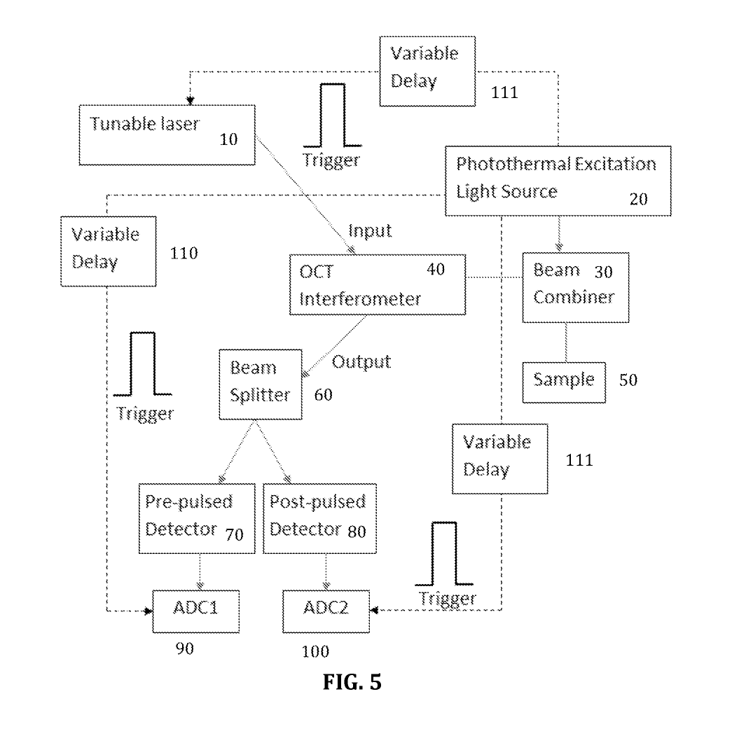

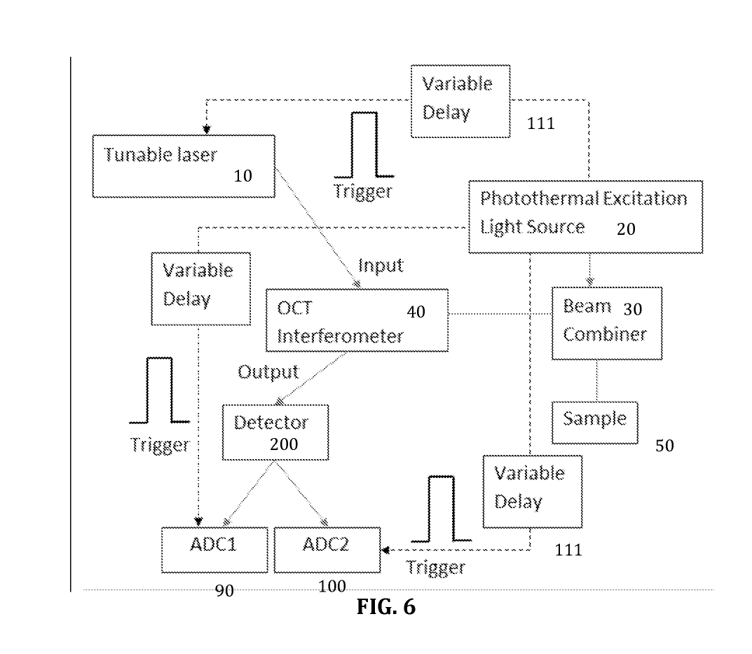

Possible synchronization approaches to acquire pre- and post-pulse OCT signals are shown in the FIGS. 5, 6, 8, 9, and 12.

The photothermal excitation light source can be used to generate trigger pulse(s) for pre- and post-pulse ADC acquisition. In one approach illustrated in FIG. 5, two distinct ADC's (70 and 80) can be used to acquire pre-pulse and post-pulse OCT interference fringe signal data. The time duration for propagation of electrical and optical signals from sample 50 to the ADCs 70 and 80 should be traced with a precision corresponding to approximately 1/10 of the time duration of the OCT probe beam emission at each single optical frequency (νᵢ). Photothermal excitation light source 20 can be used as a source of triggering pre- and post-pulse acquisition events. Each trigger for pre- and post-pulse acquisitions can be generated by the photothermal excitation light source. The time requirements for triggers arriving to ADCs and optical pulses to appropriate photodetectors for realizing timing patterns in FIGS. 3, 8, and 13 can be easily satisfied by implementing variable and constant optical and electrical delay lines as indicated in FIGS. 5-7 and 9-11.

In the scheme presented in FIG. 5 the signal splitting to the pre- and post-pulse channels takes place after optical detection of the OCT interference fringes. In the scheme presented in FIG. 5 the signal splitting to the pre- and post-pulse takes place before optical detection by means of the optical beam splitter 60. The data acquisition in pre- and post-pulsed channels 90 and 100 can be additionally regulated by electronic digital delay lines (for example 576-3706-5-ND integrated circuit delay line or similar by Micrel Inc).

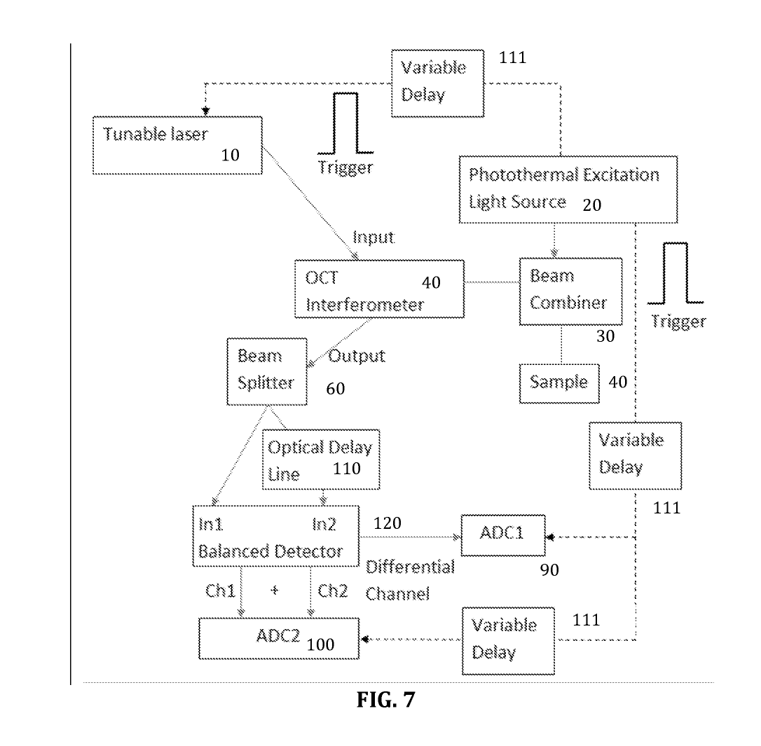

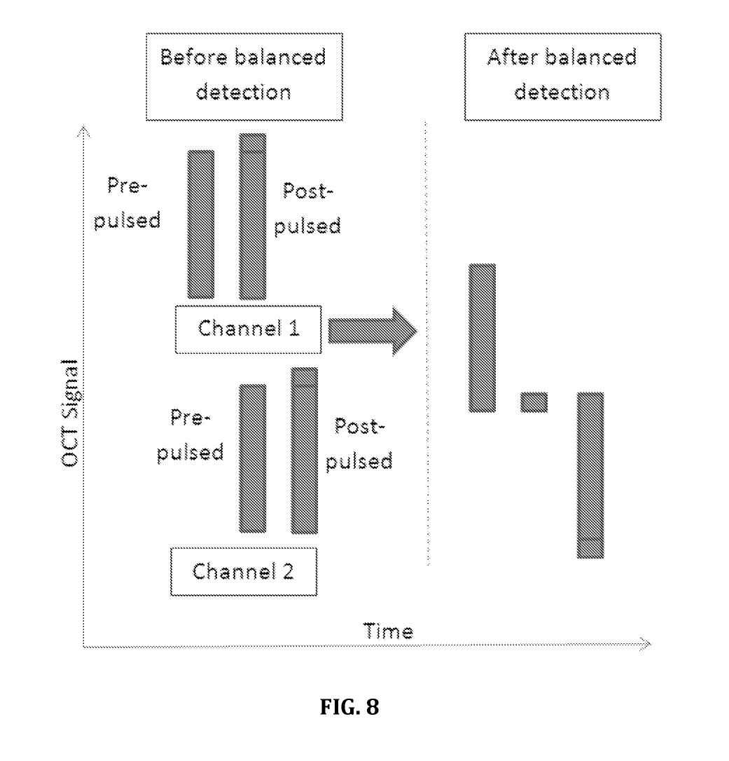

The temporally precise electronic triggering of the ADC used to acquire the OCT interference fringe signal can be relaxed by using an optical delay line 110 as showed in FIG. 7. In this approach, light from the interferometer 40 is split into two optical beams and one beam is delayed optically by the time difference between the centers of the pre- and post-pulsed OCT signal segments. Optical delay lines are well known in the art and commercial units are readily available. Next light from the OCT interferometer enters the two channels of a balanced detector 120 and differential output to the ADC1 90 is used to acquire phase information while the sum channel is used to acquire intensity OCT information. The ADC1 channel is triggered to detect only the central differential signal as shown in FIG. 8.

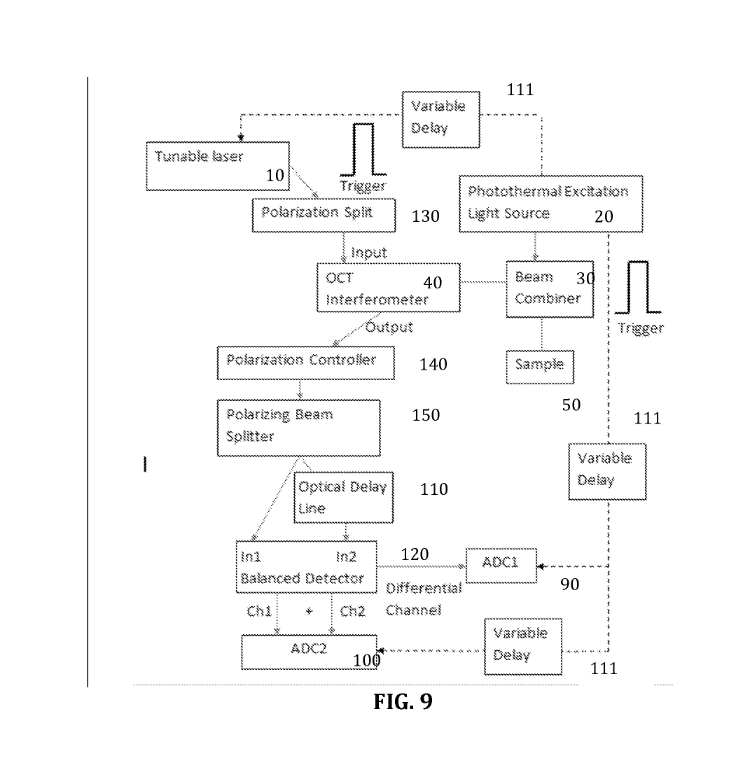

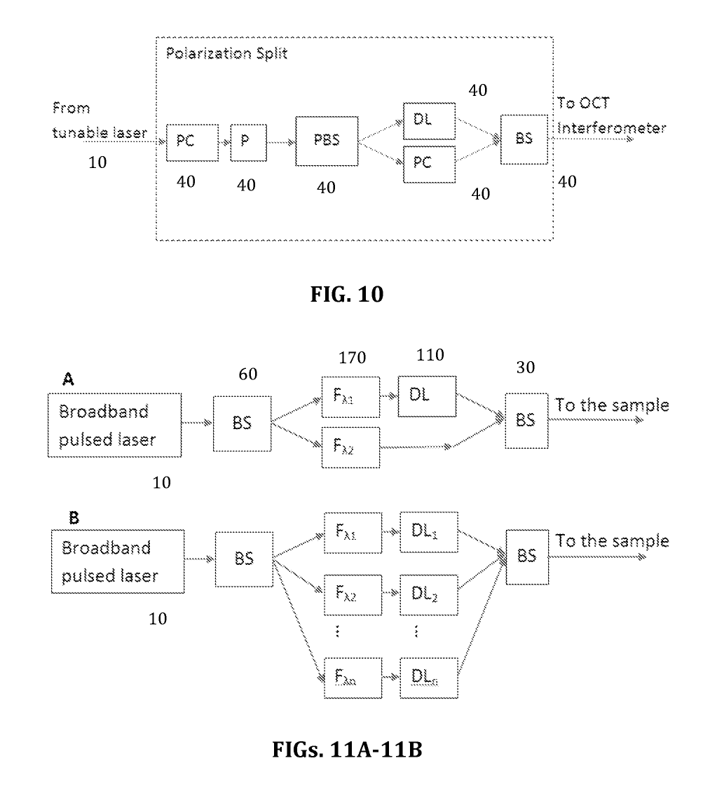

The precise triggering of both ADCs used for the intensity OCT signal and phase detection can be omitted by using an optical delay line 110 and polarized light as showed in FIG. 10. In this case, pre-pulsed OCT light is polarized orthogonally to the post-pulsed OCT light. To achieve orthogonal polarization of pre- and post-pulsed light two methods can be used by means of two types of polarization splitters. In the first approach polarization modulation is implemented using for example an electro-optical modulator. In this case, the light switched from one polarization state to an orthogonal state using an electronic trigger (not shown in the FIG. 9) originated from the photothermal excitation light source 20. In the second approach a birefringent material used to correspondingly delay one polarization state from another. A polarization-maintaining (PM) fiber, for example, can be used for this purpose. Considering a typical birefringence of a PM fiber (3×10⁻⁴), 1 km of the PM fiber provides a 1 ns delay between two orthogonal polarizations. Another embodiment of the polarization split element can be based on dividing the tunable-laser light into two beams, where polarizations and traveling optical paths (traveling time) of these beams processes independently. After which the two beams are recombined into a single beam so the resulting beam entering OCT interferometer consists of two orthogonally polarized states, where the delay between orthogonal polarization states is predetermined. One of the possible realizations of two-beam polarization split element is illustrated in FIG. 10.

The polarization control element at the output of OCT interferometer 40 can ensure each of the two beams exiting the polarization beam splitter 150 contains only pre- or post-pulsed light. In FIG. 8 before balanced detection, channel 1 contains only post-pulsed light and channel 2 contains only pre-pulsed light. Therefore after balanced detection, only the differential component is presented at the output of the balanced detector.

Light from the broadband pulsed laser (for example femtosecond laser) is split into two beams using a beam splitter (BS, FIG. 11). The first beam is passed through a notch or band bypass filter centered at specific wavelength λ₁ (for example 798 nm for oxygen sensing) and the second beam transmits through the filter centered at λ₂ (for example 765 nm for oxygen sensing). The bandwidth of the notch filters can be selected for example as 1 nm to produce pulses with a picosecond time duration, which are convenient for oxygenation sensing. The two pulses may be delayed for the desired time span using an optical delay line (DL)(e.g., optical fiber) corresponding to the time span between two neighboring optical frequencies (νᵢ and νᵢ₊₁) of the OCT source laser. The polarization states of the two beams can be also modified to investigate polarization properties of the sample. Finally two photothermal excitation beams may be combined together using beam splitter/combiner to deliver light to the sample.

In another embodiment, the OCT fixed optical frequency (νᵢ, i=1, 2, 3 etc.) can be polarized to a known state (linear, circular, or elliptical) before the photothermal pulse and the phase change induced by absorption of the photothermal excitation pulse can be measured for different polarization states.

In another embodiment, the OCT fixed optical frequency can be sampled multiple times in the post-pulse region to ascertain the evolution and heat dissipation characteristic of the sample after excitation by a photothermal excitation pulse.

In another embodiment, multiple photothermal excitation pulses can be incident on the sample in a single time period with a fixed frequency OCT probe beam. These photothermal excitation pulses can either be the same optical frequency at different time points during OCT fixed frequency emission, different optical frequencies at the same time, different frequencies at different times, or any combination thereof.

The optical frequency of the excitation light source can make or may not make a pattern to extract spectral absorption signature of specific chromophores. For example, in the case of two excitation wavelengths (optical frequencies) the excitation frequency can probe alternating OCT wavelengths so that sampling of the phase shift in response to laser excitation at the two wavelengths is decreased by two-times. The same principle is true for 3, 4, or more excitation wavelengths.

Alternatively the sum and difference signals may be generated with appropriate detector configurations (balanced detector and summing detector). The time delay between pre- and post-excitation pulses may be compensated by introducing an optical delay before the balanced and summing detector. When an optical delay is used, a single ADC may be used to acquire the difference and sum signal. Similarly, balanced fringe detection can be used to the double amplitude of OCT interference fringes and cancel intensity noise.

In certain aspects, an ultrafast optical coherence tomography apparatus can comprise (i) an akinetic laser source, (ii) a light splitting portion that splits a fixed frequency light beam emitted from the akinetic laser source into a reference beam and a OCT probe beam for irradiating a target or sample, (iii) an interferometer, and (iv) a detector comprising one or more analog to digital converters (ADCs) configured to acquire a signal generated from a fixed frequency target OCT probe beam in a time interval of less that 5 microseconds. In certain aspects, the system is configured to detect two or more OCT probe beams signals. In certain aspects, the apparatus can further comprise a photothermal excitation light source configured to provide an excitation pulse on the sample at a selected time with respect to the OCT probe beam irradiation time-interval. The apparatus can further comprise a pre-pulse detector and a post-pulse detector, or a single detector configured to acquire a signal from a pre-pulse OCT probe beam and a post-pulse OCT probe beam. In certain aspects in case of one ADC with two channels (FIG. 12) one channel continuously detects OCT interference fringe signal and the other channel continuously detects trigger events (i.e. photothermal excitation and probe beam narrow bandwidth pulses timing as well as A-scans start time). In certain aspects, the detector is a balanced detector operatively coupled to an analog to digital converter.

Various laser sources can be used to carry out aspects of the claimed invention. In a non-limiting example, the laser source such as those described in U.S. Pat. Nos. 5,365,536, 6,778,577, 5,715,266, 7,310,358, 7,339,974, 8,130,802, and the like, each of which is incorporated herein by reference in its entirety.

### III. Application of UMW-OCT

Certain embodiments are directed to methods for detecting one or more constituents comprising (a) irradiating a target site having one or more constituents using a substantially fixed optical frequency optical coherence tomography (OCT) probe beam, wherein the target is irradiated by the fixed frequency OCT probe beam for at most a 5 microsecond time interval; (b) irradiating the target site with an excitation pulse at a first wavelength that is absorbed by at least a first constituent for an excitation time interval of less than 5 microseconds during the OCT probe beam time interval, (c) measuring the OCT interference fringe signals in pre-excitation pulse and a post-excitation pulse time intervals; (d) repeating steps a-c for a multiplicity of OCT optical frequencies (νᵢ); (e) determine the optical path length change in response to photothermal excitation of the target site from the measurement of OCT interference fringe signals in pre-excitation pulse and a post-excitation pulse time intervals at the multitude of OCT optical frequencies (νᵢ), and (f) detecting a first constituent by evaluating the optical path length difference between the pre-excitation pulse optical path length and the post-excitation pulse optical path length.

In certain aspects, the relative concentrations of two or more constituents are determined by (i) irradiating the target site with an photothermal excitation pulse at a first wavelength that is absorbed by at least a first constituent and an photothermal excitation pulse at a second wavelength that is absorbed by a second constituent, for an excitation time interval of less than 5 microseconds during the OCT probe beam time interval, (ii) measuring a pre-excitation pulse and a post-excitation pulse optical path length for the target site at the two excitation wavelengths, and (iii) determining a difference between the changes in the first and second optical path length to determine the levels of the first constituent relative to the second constituent by evaluating the optical path length changes.

In certain aspects, the target is a biological target. A biological target can be a tissue, organ, cell culture and biological fluid or portions thereof. In certain aspects, the target is in an animal subject. In certain aspects, the target is a plant. In a further aspect the biological target is the retina. The biological target can be a blood vessel in the retina. In certain aspects the organ or tissue is retina, choroid, skin, tumor, epithelia, blood vessel, cervix, prostate, stomach, large intestine, small intestine, esophagus, tongue, mouth, or brain. The biological target can comprise hemoglobin and the various forms thereof. In certain aspects, the first constituent is oxygenated hemoglobin and the second constituent is deoxygenated hemoglobin, carboxy hemoglobin, sulf-hemoglobin, or methemoglobin. In certain aspects hemoglobin oxygen saturation (SaO2) is measured. In certain aspects, the first excitation radiation has a wavelength of about 800 nm. In a further aspect the second excitation radiation has a wavelength of about 765 nm.

In certain embodiments the target is a non-biological target. The non-biological target can be a pharmaceutical composition, a film, or a polymeric composition.

### IV. Examples

The following examples as well as the figures are included to demonstrate preferred embodiments of the invention. It should be appreciated by those of skill in the art that the techniques disclosed in the examples or figures represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

Ultra-fast SO₂-OCT sensitivity. Ultra-fast (12.5 ns) acquisition of phase information (δφ_{1,2}(νᵢ)) used for the SO₂ calculation allows very efficient use of the photothermal excitation energy and removes limitations of the prior SO₂-OCT (Kuranov et al., Opt Express 19(24):23831-44, 2011) associated with blood flow and tissue motion. Specifically, the acquisition time is decreased at a single lateral point (512 in depth points) from seconds to 12.8 μs with 1.7 times higher signal-to-noise ratio (SNR) and using a safe photothermal light power level (0.7 mW) incident on the cornea (prior SO₂-OCT sample power was 18 mW).

Improvement of the SNR ultra-fast (SNR_{uf}) over prior (SNRₚ) SO₂-OCT for each of the physical phenomena is summarized in the Table 1.

| 1 | 2 | 3 | 4 | 5 | 6 | 7 | 8 |
|---|---|---|---|---|---|---|---|
| Phenomena | Thermal | Excitation- | RBC's | Intensity | op noise | Excitation | Result |
| | diffusion | arteriole | motion | | | period | |
| | | overlap | | | | | |
| SNR_{uf}/SNRₚ | a₁ = 25 | a₂ = 2.6 | a₃ = 50 | a₄ = 2 | a₅ = 53 | a₆ = 5 · 10⁻⁶ | a = 1.7 |
| Equation | (rₜₕ/r_{bv})² | Sₚₜₕ/S_{bv} | 17 dB | (dₚ²P_{uf})/(d_{uf}²/Pₚ) | opₚ/op_{uf} | τ_{uf}/τₚ | a₁a₂a₃a₄a₅a₆ |
| | | | drop-off | | | | |

Column two: prior SO₂-OCT thermal diffusion radius rₜₕ=(4ατₜₕ)^{1/2}=37 μm lead to SNR decrease of (rₜₕ/r_{bv})² due to thermal diffusion energy dissipation. Here α=1.4·10⁻³ cm²/s is a water thermal diffusivity, τₜₕ=2.5 ms photothermal excitation period, r_{bv}=7.5 μm is a blood vessel lumen.

Column three: the photothermal excitation area (sₚₜₕ) of a 50 μm photothermal beam diameter in the prior SO₂-OCT was 2.6 times larger than area of the arteriole's lumen (15 μm diameter).

Column four: the op amplitude SNR (SNRₚ) decreases 1 dB per 1 mm/s of red blood cells (RBC) velocity. The 17 dB (50 times) drop-off of the SNRₚ is due to RBC velocity of 11 mm/s in 30 μm diameter arteriole and 6 mm/s Brownian RBC's motion.

Column five: intensity (I) of the ultra-fast SO₂-OCT (˜P_{uf}/d_{uf}²) is two times higher than prior SO₂-OCT intensity (˜Pₚ/dₚ²). Here P_{uf}=0.7 mW, d_{uf}=7 μm and Pₚ=18 mW, dₚ=50 μm.

Column six: prior SO₂-OCT opₚ=254 pm floor noise is due to tissue motion. It is estimated op_{uf}=4.8 pm as opₛ/N^{1/2}, where opₛ=δφ(νⱼ)λ/(4π)=153 pm estimated from uncertainty of the phase measurement at single tunable laser optical frequency δφ(νⱼ)=1/SNR_{S} and SNR_{S}=500 (averaged over 5 ADC digitization events) and N=1024 is a number of tunable laser optical frequencies per A-scan. In ultra-fast SO₂-OCT retinal motion below 3 pm during 12.5 ns measurement time (FIG. 14) will not interfere with the measurement precision. Column seven: ultra-fast SO₂-OCT's 5·10⁶ shorter time of photothermal excitation as compared with prior SO₂-OCT reduces incident fluence (Φ=Iτ) on the retina. In conclusion the new approach dramatically increases the acquisition of functional information while keeping the incident power within the safe level as required by ANSI standard for ocular laser irradiation.

1. A method for Fourier Domain optical coherence tomography comprising:
(a) exposing a first location of a target to a plurality of time resolved, narrow bandwidth optical coherence tomography (OCT) probe beams having a distinct fixed central frequency and an exposure time interval of at most 5 microseconds;
(b) acquiring an interferometric signal related to each fixed central frequency OCT probe beam;
(c) processing the acquired signals to produce an A-scan; and
(d) repeating steps a-c to produce a plurality A-scans representative of the target.

2. The method of claim 1, wherein A-scans are produce at a rate of at least 1000 A-scans per 5 milliseconds.

3. A method for Fourier-Domain photothermal optical coherence tomography comprising:
(a) exposing a target to a plurality of time resolved optical coherence tomography (OCT) probe beams, each probe beam having a distinct fixed frequency, and a probe beam exposure interval of at most 5 microseconds;
(b) exposing the target to an excitation pulse of less than 5 microseconds during the probe beam exposure interval;
(c) acquiring an interferometric signal related to each probe beam exposure interval before the excitation pulse and after the excitation pulse; and
(c) processing the acquired signals.

4. The method of claim 3, wherein the interferometric signal is acquired continuously.

5. The method of claim 3, further comprising a plurality of excitation pulses during the probe beam exposure interval and acquiring a signal before and after each excitation pulse.

6. A method for detecting one or more constituents comprising:
(a) irradiating a target site having one or more constituents using a fixed frequency optical coherence tomography (OCT) probe beam, wherein the target is irradiated by the fixed frequency probe beam for at most a 5 microsecond time interval;
(b) irradiating the target site with an excitation pulse at a first wavelength that is absorbed by at least a first constituent for an excitation time interval of less than 1 microsecond during the OCT probe beam time interval,
(c) measuring a pre-excitation pulse and a post-excitation pulse optical path length for the target site, and
(d) detecting a first constituent by evaluating the optical path length difference between the pre-excitation pulse optical path length and the post-excitation pulse optical path length.

7. The method of claim 6, wherein the relative concentrations of two or more constituents are determined by (i) irradiating the target site with an excitation pulse at a first wavelength that is absorbed by at least a first constituent and an excitation pulse at a second wavelength that is absorbed by a second constituent, for an excitation time interval of less than 1 microsecond during the OCT probe beam time interval, (ii) measuring a pre-excitation pulse and a post-excitation pulse optical path length for the target site at the two excitation wavelengths, and (iii) determining a difference between the changes in the first and second optical path length to determine the levels of the first constituent relative to the second constituent by evaluating the optical path length changes.

8. The method of claim 7, wherein the target is a biological target.

9. The method of claim 8, wherein the biological target is a tissue, organ, or biological fluid.

10. The method of claim 9, wherein the target is in a subject.

11. The method of claim 8, wherein the biological target is the retina.

12. The method of claim 11, wherein the biological target is a blood vessel in the retina.

13. The method of claim 8, wherein the biological target is a portion of an organ or tissue.

14. The method of claim 13, wherein the organ or tissue is retina, choroid, skin, tumor, epithelia, blood vessel, cervix, prostate, stomach, large intestine, small intestine, esophagus, tongue, mouth, or brain.

15. The method of claim 14, wherein the biological target comprises hemoglobin.

16. The method of claim 8, wherein the first constituent is oxygenated hemoglobin.

17. The method of claim 8, wherein the second constituent is deoxygenated hemoglobin, carboxy hemoglobin, sulf-hemoglobin, or methemo hemoglobin.

18. The method of claim 8, wherein hemoglobin oxygen saturation (SaO₂) is measured.

19. The method of claim 8, wherein the first excitation radiation has a wavelength of about 800 nm.

20. The method of claim 8, wherein the second excitation radiation has a wavelength of about 765 nm.

21. The method of claim 6, wherein the target is a non-biological target.

22. The method of claim 21, wherein the non-biological target is a pharmaceutical composition, a film, or a polymeric composition.

23. An ultrafast optical coherence tomography apparatus comprising:
(i) laser source having a pulse train of narrow band beams at fixed central frequency, (ii) a light splitting portion that splits a fixed frequency light beam emitted from the laser source into a reference beam for irradiating a reference mirror and a probe beam for irradiating a target, (iii) an interferometer, and (iv) a detector comprising one or more analog to digital converters configured to acquire a first signal generated from a first fixed frequency target probe beam in a time interval of less that 5 microseconds and at least a second signal generated from a second fixed frequency probe beam in a time of less that 5 microseconds, wherein the time between irradiation of a target with the first fixed frequency probe beam and irradiation of the target by the second fixed frequency probe beam is less than 5 nanoseconds.

24. The apparatus of claim 23, further comprising an excitation beam source configured to provide an excitation pulse with a probe beam irradiation interval.

25. The apparatus of claim 24, further comprising a pre-pulse detector and a post-pulse detector.

26. The apparatus of claim 23, further comprising a balanced detector operatively coupled to an analog to digital converter.

27. A method for constructing an optical coherence tomography probe beam comprising:
(a) directing a light beam from a broadband pulsed optical source to a system of spatially, temporally, or dynamically changing narrow bandwidth reflectors to provide a plurality of spatially resolved beams having a distinct fixed central frequency;
(b) directing the plurality of spatially resolved beams through a dispersive optical system so the different optical frequencies travel with different speed for a fixed distance and acquire different time delays.
